(19) United States
(12) Patent Application Publication
(10) Pub. No.: US 2007/0218048 A1
(43) Pub. Date: Sep. 20, 2007

(54) Sfec, a Sperm Flagellar Energy Carrier Protein

(76) Inventors: Young-Hwan Kim, Charlottesville, VA (US); John C. Herr, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/593,532

(22) PCT Filed: Mar. 17, 2005
(86) PCT No.: PCT/US05/08906

§ 371 Date: Sep. 18, 2006

(30) Foreign Application Priority Data
Mar. 17, 2004 (US) 60554085
Sep. 30, 2004 (US) 60614817

(51) Int. Cl. A61K 39/395; A61K 38/00; C07H 21/04; C07K 14/00; C07K 16/18; C12N 15/00; C12N 5/06; G01N 33/53

(52) U.S. Cl. 424/130.1; 424/185.1; 435/320.1; 435/325; 435/7.1; 514/2; 514/44; 530/350; 530/387.1; 530/387.9; 536/23.1

(57) ABSTRACT

The present invention is directed to a sperm flagellar energy carrier, SFEC, antibodies specific for the SFEC and the use of the SFEC protein to identify antagonists of SFEC activity. SFEC is believed to be essential for sperm motility, is localized to a specific region of the sperm, and thus antagonists of SFEC activity are anticipated to have utility as contraceptive agents.

### CROSS REFERENCE TO RELATED APPLICATIONS

This application is entitled to priority pursuant to 35 U.S.C. § 119(e) to U.S. provisional patent application Nos. 60/554,085, filed on Mar. 17, 2004, and 60/614,817, filed on Sep. 20, 2004.

### US GOVERNMENT RIGHTS

This invention was made with United States Government support under Grant No. TW 00654, awarded by National Institutes of Health. The United States Government may have certain rights in the invention.

### BACKGROUND

Sperm motility is dependent on a functional flagellum. A sperm flagellum consists of several cytoskeletal components including the fibrous sheath. The fibrous sheath is a unique cytoskeletal component in the principal-piece segment of the mammalian sperm flagellum. The fibrous sheath surrounds the axoneme (a motile sliding apparatus) and outer dense fibers and defines the extent of the principal piece region of the sperm flagellum. It consists of two longitudinal columns connected by closely arrayed semicircular ribs that assemble from distal to proximal throughout spermiogenesis. A comprehensive review of the protein composition of the fibrous sheath was recently written (Eddy et al., Microsc Res Tech. 2003, 61:1:103-15).

The concept that the fibrous sheath serves as a scaffold for glycolysis is based upon the light and electron microscopic localization of two enzymes of the glycolytic pathway, hexokinase 1 and glyceraldehyde 3 phosphate dehydrogenase to the ribs and longitudinal columns of the fibrous sheath.

The principal piece is the longest domain of the sperm tail and although lacking mitochondria, it contains major cytoskeletal elements of the flagellum, including axoneme, outer dense fibers and fibrous sheath (FS), the latter structure being restricted to the principal piece. Past reports localized two glycolytic enzymes, hexokinase and GAPDH, to the fibrous sheath, and a role for FS in glycolysis has been posited. However, little is currently known about energy production in the principal piece and there is at present no mechanism to explain energy translocation to dynein ATPases which function as force generating motors along the distal flagella.

There are several functions for the fibrous sheath that have emerged to date: 1) the fibrous sheath functions as a protective girdle for the sperm axoneme while maintaining flagellar flexibility and affecting the plane of the flagellar beat; 2) the fibrous sheath, through its A kinase anchoring proteins AKAP 3 and AKAP4, serves as a scaffold for enzymes involved in signal transduction including protein kinase A, the Rho signaling pathway through rhoporrin and rhophilin, and presumably calcium signaling via CABYR; and 3) the fibrous sheath anchors enzymes involved in the glycolytic pathway.

The concept that the fibrous sheath serves as a scaffold for glycolysis is based upon the light and electron microscopic localization of two enzymes of the glycolytic pathway, hexokinase 1 and glyceraldehyde 3 phosphate dehydrogenase to the ribs and longitudinal columns of the fibrous sheath.

There is a need for better means of contraception and a need for rapid, economical, and accurate diagnostic tests for sperm motility and fertility problems. The present invention satisfies these needs.

### SUMMARY OF VARIOUS EMBODIMENTS OF THE INVENTION

As described herein additional glycolytic pathway enzymes have now been associated with the fibrous sheath providing further evidence that the fibrous sheath serves as a scaffold for glycolysis. Furthermore, applicants have now discovered a novel, sperm specific fibrous sheath protein, that is believed to function as an adenine nucleotide translocase, and thus has been designated sperm flagellar energy carrier (SFEC). In addition, applicants have now discovered that the novel, sperm specific fibrous sheath protein, is located in the principal piece of the sperm tail, but not in the midpiece.

The present invention is directed to a sperm flagellar energy carrier protein (SFEC), antibodies specific for SFEC and nucleic acid sequences encoding said protein, as well as compositions comprising such compounds. SFEC is believed to be essential for sperm motility, and thus antagonists of SFEC activity are anticipated to have utility as contraceptive agents. Compositions comprising the proteins, amino acid sequences, nucleic acid sequences, or antibodies of the present invention fertility.

Various aspects and embodiments of the invention are described in further detail below.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 represents a photographic image of the isolated human fibrous sheath prepared by mechanical and chemical means as visualized using transmission electron microscopy.

FIG. 2 represents an SDS-PAGE image of isolated human fibrous sheath proteins (FS). The FS nomenclature is indicated on the right and is summarized in Table 1.

FIG. 3 schematically summarizes the peptide sequences identified from microsequencing the C265 band and the human and mouse associated protein sequences.

FIG. 4 represents a Northern blot of poly A RNA isolated from human spleen, thymus, prostate, testis ovary, small intestine, colon and leukocytes probed with 32P labeled SFEC cDNA, demonstrating that SFEC is a testis specific protein. SFEC cDNA corresponding to full length of ORF was radiolabeled with 32P and hybridized to 2 μg poly(A)+ mRNAs revealing 2.4-kb message only in testicular RNA. Size of molecular weight markers is indicated at left. Human Northern blot used for SFEC cDNA was stripped and hybridized with 32P-labeled cDNA of β-actin as a positive control.

FIG. 5 represents a dot blot analysis (upper panel) of RNA from 76 different human tissues, again showing that SFEC is a testis specific protein. The upper panel represents an image of a dot blot, while the lower panel is a schematic of a human multiple tissue expression (MTE) array summarizing the analysis. The lower panel is demarcated by 12 columns and rows A to H. The human multiple tissue expression array contained normalized loadings of poly A+ RNA from 76 different human tissues (see diagram at right) probed with 32P-labeled human SFEC cDNA. E. coli DNA was also hybridized. GenBank Submission (see attached) GenBank has provided GenBank accession numbers Human: AY550240 and Mouse: AY550241.

FIG. 6, comprising upper, middle, and lower panels, is a schematic representation of the functional domains of SFEC.

FIG. 7 is a schematic representation of the alignment of the amino acid sequences of SFEC with other human proteins having similar domains.

FIG. 8 is a schematic representation summarizing human fibrous sheath proteins involved in energy production, their tissue distribution, and their gene loci.

FIG. 9, comprising FIGS. 9A, 9B, and 9C, represents an electrophoretic analysis of SFEC protein. FIG. 9A is an image of a Coomassie blue stained gel of induced (center lane) and uninduced (right lane) truncated recombinant SFEC (117 amino acid residues) recombinant protein (arrow) expressed in BLR (DE3) host cells. The arrow indicates the SFEC stained band. The left lane (control) is the molecular weight marker peptide lane. FIG. 9B is an image of a Western blot analysis of induced (left lane) and uninduced (right lane) recombinant SFEC using an anti-histidine antibody. FIG. 9C depicts an image of affinity purified recombinant SFEC (arrow) stained by SYPRO Ruby stain.

FIG. 10, comprising FIGS. 10A, 10B, and 10C, represents a Western blot analysis of recombinant SFEC, human sperm, and isolated FS proteins, using an anti-SFEC antibody. FIG. 10A is an image of a Western blot analysis using an antibody against SFEC to detect electrophoresed recombinant SFEC (recSFEC) comparing Post-immune serum (left lane) and Pre-immune serum (right lane). FIG. 10B is an image of a Western blot analysis using an antibody against SFEC comparing Post-immune serum (left lane) and Pre-immune serum (right lane) on human sperm. FIG. 10C is an image of a Western blot analysis using an antibody against SFEC comparing Post-immune serum (left lane) and Pre-immune serum (right lane) on electrophoresed FS protein. Post immune serum recognized recombinant SFEC (A) and three bands at 38, 32 and 20 kDa on the human sperm proteins (B). The FS was recognized at the 32 kDa (C) which is initially identified as SFEC from mass spectrometry. Preimmune serum did not recognize any protein of human sperm (B), FS(C), or recombinant SFEC (A).

FIG. 11, comprising FIGS. 11A, 11B, 11C, 11D, 11E, and 11F, represents an indirect immunofluorescence analysis of human swim-up sperm using rat serum against recombinant human SFEC protein, localizing SFEC to the principal piece of the flagellum of human sperm. FIG. 11A represents an image of a phase contrast micrograph corresponding to FIG. 11B (FITC only), FIG. 11C (FITC+phase), and FIG. 11D (FITC+DAPI). FIG. 11E is an image of a phase contrast micrograph of sperm. FIG. 11F is an image of FITC, corresponding with FIG. 11E, of a control experiment using pre-immune serum. Large arrow-principal piece; Small arrow-midpiece. Approximately 50% of sperm were recognized by the SFEC antibody which is directed against N-terminal 120 amino acids

### DETAILED DESCRIPTION OF THE INVENTION

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

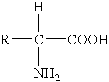

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies, and fragments thereof.

As used herein, the term “SFEC antibody” refers to an antibody that specifically binds to the amino acid sequence of SEQ ID NO: 2 or SEQ ID NO: 4, or fragments thereof.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

“Biologically active,” as used herein with respect to SFEC proteins, peptides, fragments, derivatives, homologs and analogs means that the proteins, peptides, fragments, derivatives, homologs and analogs have the ability to function as an SFEC protein as described herein.

As used herein, the term “biologically active fragments” or “bioactive fragment” of an SFEC polypeptide encompasses natural or synthetic portions of the full-length protein that are capable of specific binding to their natural ligand.

“Complementary,” as used herein, refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “control” cell is a cell having the same cell type as a test cell or sample cell. The control cell is obtained from a normal subject or a subject not being treated with a compound used to treat a test subject. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell. The control cell may also be obtained from another source or similar source other than the test cell group or a test subject, where the test cell is obtained from a subject suspected of having a disease or disorder for which the test is being performed. The control cell may be tested in vitro or in vivo.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

The terms “detect” and “identify” are used interchangeably herein.

An “effective amount” or “therapeutically effective amount” of a compound is that amount of compound which is sufficient to provide a beneficial effect to the subject to which the compound is administered.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression,” as used with respect to SFEC mRNA, refers to transcription of a nucleic acid comprising a nucleic acid sequence encoding SFEC mRNA, resulting in synthesis of SFEC mRNA. “Expression,” as used with respect to an SFEC protein, or homolog, derivative, or fragment thereof, refers to translation of SFEC mRNA, resulting in protein synthesis of an SFEC protein, or homolog, derivative, or fragment thereof.

A “fragment” or “segment” is a portion of an amino acid sequence, comprising at least one amino acid, or a portion of a nucleic acid sequence comprising at least one nucleotide. The terms “fragment” and “segment” are used interchangeably herein. A fragment of a protein or peptide may ordinarily be a least about 20 amino acids in length.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

A “host cell” that comprises a recombinant polynucleotide is referred to as a “recombinant host cell.” A gene which is expressed in a recombinant host cell wherein the gene comprises a recombinant polynucleotide, produces a “recombinant polypeptide.”

The term “inhibit,” as used herein, means to suppress or block an activity or function by at least about ten percent relative to a control value. Preferably the activity is inhibited by about 50% compared to a control value, more preferably by about 75%, and even more preferably by about 95%. “Inhibit,” “block,” and “suppress” are used interchangeably herein.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

“Modified” compound, as used herein, refers to a modification or derivation of a compound, which may be a chemical modification, such as in chemically altering a compound in order to increase or change its functional ability or activity.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Operably linked” refers to a juxtaposition wherein the components are configured so as to perform their usual function. Thus, control sequences or promoters operably linked to a coding sequence are capable of effecting the expression of the coding sequence.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR—linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂ NR—) linkage, a urea (—NHC(O)NH—) linkage, a —CH₂-secondary amine linkage, or with an alkylated peptidyl linkage (—C(O)NR—) wherein R is C₁-C₄ alkyl;

2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;

3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

“Plurality” means at least two.

A “polylinker” is a nucleic acid sequence that comprises a series of three or more closely spaced restriction endonuclease recognitions sequences.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

The term “non-native promoter” as used herein refers to any promoter that has been operably linked to a coding sequence wherein the coding sequence and the promoter are not naturally associated (i.e. a recombinant promoter/coding sequence construct).

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

A “recombinant polypeptide” is one which is produced upon expression of a recombinant polynucleotide.

“A regulator of sperm flagellar energy carrier protein,” as used herein, refers to a compound which regulates SFEC expression, levels, or function. In one aspect the regulator may be an inhibitor of SFEC expression, levels, or function. In another aspect, the regulator may stimulate or increase SFEC expression, levels, or function.

A “sample,” as used herein, refers to a biological sample from a subject, including normal tissue samples, tumor tissue samples, blood, urine, semen, or any other source of material obtained from a subject which contains a compound or cells of interest.

As used herein, “SFEC” represents Sperm Flagellar Energy Carrier Protein. SFEC can be used interchangeably with “testis adenine nuclear transporter” (“tANT”).

As used herein, “SFEC activity” refers to functions or properties of SFEC, such as, but not limited to, its ability to function as an adenine nuclear transporter.

An “SFEC-associated disease or disorder,” as used herein, refers to a disease or disorder in which there is an association with a mutated or defective SFEC gene or protein, or with aberrant expression or regulation of SFEC expression, or with aberrant levels of SFEC.

As used herein, the term “SFEC polypeptide” and like terms refer to a polypeptide comprising an amino acid sequence selected from the group consisting of SEQ ID NO: 2, SEQ ID NO: 4, and fragments thereof.

A “subject,” as used herein, can be a human or non-human animal. Non-human animals include, for example, livestock and pets, such as ovine, bovine, equine, porcine, canine, feline and murine mammals, as well as reptiles, birds and fish. Preferably, the subject is a human. A “subject” of diagnosis or treatment is a human or non-human animal.

As used herein, a “substantially similar amino acid sequence” refers to a peptide or a portion of a peptide which has an amino acid sequence identity or similarity to a reference peptide of at least about 70%. Preferably, the sequence identity is at least about 75%, more preferably at least about 80%, more preferably at least about 85%, particularly preferably at least about 90%, and more particularly preferably at least about 95%, and most preferably at least about 98%. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially similar nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially similar nucleic acid sequence encodes the peptide encoded by the reference nucleic acid sequence. The percentage of identity between the substantially similar nucleic acid sequence and the reference nucleic acid sequence is at least 70%. Preferably, the sequence identity is at least about 75%, more preferably at least about 80%, more preferably at least about 85%, particularly preferably at least about 90%, and more particularly preferably at least about 95%, and most preferably at least about 98%. Substantial similarity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al. (1984), Nucl. Acids Res. 12:387), and the BLASTN or FASTA programs (Altschul et al. (1990), supra). The default settings provided with these programs are suitable for determining substantial similarity of nucleic acid sequences for purposes of the present invention.

As used herein, the term “treating” includes prophylaxis of the specific disease, disorder, or condition, or alleviation of the symptoms associated with a specific disorder or condition and/or preventing or eliminating said symptoms. A “prophylacetic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

As used herein, a transgenic cell is any cell that comprises a nucleic acid sequence that has been introduced into the cell in a manner that allows expression of a gene encoded by the introduced nucleic acid sequence.

The terms to “treat” or “treatment,” as used herein, refer to administering an agent or compound to reduce the frequency with which symptoms of an SFEC-associated disease disorder are experienced, to reduce the severity of symptoms, or to prevent symptoms from occurring. Treatment can restore the effect of SFEC function or activity which has been lost or diminished in an SFEC-associated disorder. “Treatment” also includes methods of regulating SFEC for contraceptive purposes.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### EMBODIMENTS OF THE INVENTION

Fertility Requires Sperm Motility and Consequently ATP Production

Oxidative phosphorylation in mitochondria is the most efficient way to produce ATP, but in the case of spermatozoa, the mitochondria are localized solely in the sperm mid piece and yet the flagella extends another 40 um or so beyond the base of the mid piece. This raises the question of how ATP is generated and made available for the dynein-ATPases of the mitochondrion-free part of the flagellum (principal piece).

The results from one-dimensional SDS-PAGE revealed that the fibrous sheath contains at least 17 distinct Coomassie staining protein bands. These bands were assigned a nomenclature of C253-C269, and each band was cored and microsequenced by tandem mass spectrometry. The results indicate that the isolated fibrous sheath preparation contained many proteins that had been previously characterized as fibrous sheath components including roporrin, AKAP3, AKAP4, GST mu, and GAPDH-2. These findings confirmed the purity of the isolated fibrous sheath preparation. However, more significantly, microsequencing of isolated human fibrous sheath also revealed the presence of five glycolytic proteins, not previously reported to be associated with the fibrous sheath. These enzymes are aldolase A, sorbitol dehydrogenase, lactate dehydrogenase, triosphosphate iosmerase, pyruvate kinase. The addition of 5 new components to the 2 previously known glycolytic enzymes contained in the human fibrous sheath conclusively establishes glycolysis as a process occurring in the principal piece of the sperm flagellum, independent of ATP generation in the mitochondria. Glycolysis is an essential metabolic pathway that may proceed in the absence of oxygen to generate ATP. Accordingly, these findings demonstrate that the fibrous sheath is a flagellar sub-compartment for the glycolytic pathway to generate ATP under anaerobic condition.

Bioinformatic analysis of the five glycolytic peptides that were obtained from the human fibrous sheath indicate that the glycolytic enzymes represent the somatic form each enzyme, with the exception of the testis specific form of lactate dehydrogenase, LDHC. Although testis isoforms of triose phosphate isomerase have been identified in humans (Strausberg et al., Proc. Natl. Acad. Sci. U.S.A. 99 (26), 16899-16903 (2002)), the peptides identified in the fibrous sheath represent the somatic form of TPI rather than the testis isoform. This indicates the fibrous sheath glycolytic machinery is comprised of two subsets of glycolytic enzymes: testis specific as well as somatic isoforms.

As described herein, the human SFEC protein is 315 amino acids in length, has a molecular weight of 35021.78 daltons, an isoelectric point of 10.4632, a charge of 24.5 and an average residue weight of 111.180.

The nucleotide sequence of the human SFEC mRNA covers 1727 bp including an open reading frame that yields a protein of 315 amino acid residues. The gene structure of SFEC spans approximately 43.8 kb divided into 6 exons and 5 introns. The human SFEC gene was localized to chromosome 4q28.2, while murine SFEC was localized to chromosome 3B. The other known human ADP/ATP carrier proteins in the same family such as heart/skeletal muscle isoformT1 (ANT 1) and liver isoformT2 (ANT 3) were localized to chromosome 4 q35.1 and chromosome X p22.33, respectively. Fibroblast isoform (ANT2) was localized to chromosome X q24. From this evidence indicating the presence of an uncharacterized unique gene the C265 protein is believed to be a novel member of the family of ADP/ATP Carrier Proteins, also known as the ADP/ATP Translocase, or alternatively, Adenine Nucleotide Translocator or ANT. Since the C265 protein described herein was isolated from the fibrous sheath and because a role in signal transduction or glycolysis or both is likely, the novel protein has been designated as a sperm flagellar energy carrier protein or SFEC. At this time, it is not yet apparent if SFEC functions as an ATP reserve (e.g., storage/sink) or as an ATP carrier which shuttles ATP to the axoneme.

It is known that testis specific isoforms (Hk1-sa, Hk1-sb and Hk1-sc) of hexokinase 1 are produced from a single somatic gene Hk1 by alternative splicing. In contrast the testis specific form of GAPDH, GAPDS, is encoded by a unique gene locus Gapds in mouse and GAPDH2 in humans. Thus, of the two known glycolytic enzymes localized in the flagellum, testis specific isoforms exist, and these are generated by either alternative splicing or expression of unique genes. The bioinformatic analysis of the peptides isolated from the human fibrous sheath indicates that they are all somatic isoforms and do not represent testis specific isoforms, although such forms have been described for triose phosphate isomerase and LDHC, the germ cell-specific member of the lactate dehydrogenase family. This supports the fibrous sheath as being comprised of testis specific and somatic members of the glycolytic enzyme families.

The nucleic acid sequences of human and mouse SFEC are designated as SEQ ID NO: 1 and SEQ ID NO:3, respectively, and the deduced human and mouse amino acid sequences are designated as SEQ ID NO:2 and SEQ ID NO:4, respectively. The human and mouse SFEC share 83% identity and 89% similarity of protein sequences.

In accordance with one embodiment of the present invention a purified polypeptide is provided comprising the amino acid sequence of SEQ ID NO:2 or SEQ ID NO:4, or an amino acid sequence that differs from SEQ ID NO:2 or SEQ ID NO:4 by 1-5 conservative amino acid substitutions, or homologs, fragments, or derivatives thereof. In one embodiment, the purified polypeptide comprises an amino acid sequence that differs from SEQ ID NO:2 by 20 or less conservative amino acid substitutions, and in another embodiment by 10 or less conservative amino acid substitutions. Alternatively, the polypeptide may comprise an amino acid sequence that differs from SEQ ID NO:2 or SEQ ID NO 4 by 1 to 5 alterations, wherein the alterations are independently selected from a single amino acid deletion, single amino acid insertion and conservative amino acid substitutions. In one embodiment, the purified polypeptide comprises the amino acid sequence of SEQ ID NO: 2.

The polypeptides of the present invention may include additional amino acid sequences to assist in the stabilization and/or purification of recombinantly produced polypeptides. These additional sequences may include intra- or inter-cellular targeting peptides or various peptide tags known to those skilled in the art. In one embodiment, the purified polypeptide comprises an amino acid of SEQ ID NO:2 and a peptide tag, wherein the peptide tag is linked to SEQ ID NO:2. In another embodiment, the purified polypeptide comprises an amino acid of SEQ ID NO:4 and a peptide tag, wherein the peptide tag is linked to SEQ ID NO:4. Suitable expression vectors for expressing such fusion proteins and suitable peptide tags are known to those skilled in the art and commercially available. In one embodiment the tag comprises a His tag.

The present invention also encompasses isolated nucleic acids comprising nucleic acid sequences which encode SFEC. In one embodiment, a purified nucleic acid is provided comprising the sequence of SEQ ID NO:1, SEQ ID NO:3 or a derivative, homolog, or fragment of SEQ ID NO:1 or SEQ ID NO:3.

The present invention also encompasses recombinant human SFEC gene constructs. In one embodiment, the recombinant gene construct comprises a non-native promoter operably linked to a nucleic acid sequence comprising SEQ ID NO:1 or SEQ ID NO:3. The non-native promoter is preferably a strong constitutive promoter that enables expression of the gene construct in a predetermined host cell. These recombinant gene constructs can be introduced into host cells to produce transgenic cell lines that synthesize the SFEC gene products. Host cells can be selected from a wide variety of eukaryotic and prokaryotic organisms, and two preferred host cells are E. coli and yeast cells.

In accordance with one embodiment, a nucleic acid sequence comprising SEQ ID NO: 1 or SEQ ID NO: 3 is inserted into a eukaryotic or prokaryotic expression vector in a manner that operably links the gene sequence to the appropriate regulatory sequences, and SFEC is expressed in the eukaryotic or prokaryotic host cell. In one embodiment the gene construct comprises the nucleic acid sequence of SEQ ID NO: 1 or SEQ ID NO: 3 operably linked to a eukaryotic promoter. Suitable eukaryotic host cells and vectors are known to those skilled in the art. The baculovirus system is also suitable for producing transgenic cells and synthesizing the SFEC genes of the present invention. One aspect of the present invention is directed to transgenic cell lines that express human SFEC and fragments of the human SFEC coding sequence.

In one embodiment the introduced nucleic acid is sufficiently stable in the transgenic cell (i.e. incorporated into the cell's genome, or present in a high copy plasmid) to be passed on to progeny cells. The cells can be propagated in vitro using standard cell culture procedure, or in an alternative embodiment, the host cells are eukaryotic cells and are propagated as part of a non-human animal, including for example, a non-human transgenic animal. In one embodiment the transgenic cell is a human cell propagated in vitro and comprises the nucleic acid sequence of SEQ ID NO: 1 or SEQ ID NO: 3.

The present invention also encompasses a method for producing human and mouse SFEC. The method comprises the steps of introducing a nucleic acid sequence comprising a sequence that encodes the human or mouse SFEC into a host cell, and culturing the host cell under conditions that allow for expression of the introduced SFEC gene. In one embodiment the promoter is a conditional or inducible promoter, alternatively the promoter may be a tissue specific or temporal restricted promoter (i.e. operably linked genes are only expressed in a specific tissue or at a specific time). The synthesized SFEC can be purified using standard techniques and used in high throughput screens to identify inhibitors of SFEC activity. Alternatively, in one embodiment the recombinantly produced SFEC polypeptides, or fragments thereof are used to generate antibodies against the human or mouse SFEC. The recombinantly produced SFEC proteins can also be used to obtain crystal structures. Such structures would allow for crystallography analysis that would lead to the design of specific drugs to inhibit SFEC function.

Consistent with SFEC's sequence similarity to other known ADP/ATP carrier proteins and its testis specific expression, the new fibrous sheath protein SFEC is predicted to have a function related to the diffusion of ATP (produced by glycolysis) through the principal piece of the flagellum. SFEC may function either as an energy carrier protein for sperm motility or alternatively, as a reservoir of ATP or ADP. Accordingly, this protein represents a target for a small molecule inhibitor that is anticipated to have a contraceptive effect. Such an inhibitor might be effective as either a male contraceptive or an intravaginal spermicidal product.

In accordance with one embodiment of the present invention, a method is provided for identifying and isolating agents that stimulate or inhibit SFEC activity and thus serve as contraceptive agents. In one aspect, the SFEC activity is glycolytic activity. In one aspect, the agent identified inhibits glycolysis. More particularly, in one embodiment, agents will be screened for their ability to interfere with SFEC's ability to bind ADP and/or ADP. Small molecules that are capable of penetrating the sperm plasma membrane will be highly desirable. In addition the small molecule inhibitors should not be toxic to somatic cells. Isolated SFEC inhibitors will be used in accordance with the present invention either alone or in conjunction with other contraceptive agents to prevent unintended pregnancies. In one aspect, a compound identified by the method of the invention regulates SFEC adenine nucleotide translocase function.

In accordance with another embodiment of the present invention, an antigenic composition is provided comprising a purified peptide comprising amino acid sequence SEQ ID NO:2, SEQ ID NO:4, or antigenic fragments thereof. The composition can be combined with a pharmaceutically acceptable carrier or adjuvant and administered to a mammalian species to induce an immune response. Such antigenic compositions have utility for raising antibodies against the SFEC protein for use in diagnostic purposes, or in one embodiment for use in contraceptive vaccine formulations. The vaccines of the invention may be multivalent or univalent. Multivalent vaccines are made from recombinant viruses/vectors that direct the expression of more than one antigen.

Suitable preparations of antigenic compositions include injectables, either as liquid solutions or suspensions; solid forms suitable for solution (or suspension) in liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water saline, dextrose, glycerol, ethanol, or the like and combinations thereof. In addition, if desired, the antigenic composition may also include minor amounts of auxiliary substances such as wetting or emulsifying agents, pH buffering agents, and/or adjuvants which enhance the effectiveness of the vaccine.

Examples of adjuvants which may be effective, include, but are not limited to: mineral gels, e.g., aluminum hydroxide, surface active substances such as lysolecithin, pluronic polyols; polyanions; peptides; oil emulsions; alum, and MDP; N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutarninyl-L-alanine-2-(1′-2′-dipalmitoyl-sn-glycero-3-hydroxyphosphoryloxy)-ethylamine, aluminum hydroxide.

The polypeptides may be formulated into the compositions as neutral or salt forms. Pharmaceutically acceptable salts include the acid addition salts (formed with free amino groups of the peptide) and which are formed with inorganic acids, such as, for example, hydrochloric or phosphoric acids, or organic acids such as acetic, oxalic, tartaric, maleic, and the like. Salts formed with free carboxyl groups may also be derived from inorganic bases, such as, for example, sodium potassium, ammonium, calcium, or ferric hydroxides, and such organic bases as isopropylamine, trimethylamine, 2-ethylamino ethanol, histidine, procaine and the like.

The present invention also encompasses antagonists and agonists, including compounds or nucleotide constructs that inhibit expression or the activity of human SFEC (i.e. transcription factor inhibitors, antisense, interference RNA and ribozyme molecules, antisense oligonucleotides, or gene or regulatory sequence replacement constructs) as well as antibodies that interfere with the activity of SFEC. Antagonists of SFEC activity can be used as contraceptive agents. In accordance with one embodiment a method for identifying antagonists of SFEC activity is provided. The method comprises the steps of contacting an SFEC protein, in the presence and absence of a potential SFEC antagonist, with ATP or ADP or other adenosine derivative and identifying antagonists of SFEC activity based on the ability of said potential SFEC antagonist to decrease binding of ATP or ADP or other adenosine derivative to SFEC. In one embodiment the SFEC protein comprises an amino acid sequence of SEQ ID NO: 2. The present invention also encompasses a method of providing contraception to mammalian species, said method comprising the steps of contacting mammalian sperm cells with a composition comprising an inhibitor of SFEC activity.

In accordance with one embodiment of the present invention, an antibody is provided that specifically binds to the human and/or mouse SFEC polypeptide (i.e. SEQ ID NO: 2 or 4), or to homologs, derivatives, or fragments thereof. In accordance with one embodiment, an antibody is provided that specifically binds to the polypeptide of SEQ ID NO: 2, or to homologs, derivatives, or fragments thereof. In one aspect, the antibody inhibits the function or activity of SFEC, or homologs, derivatives, or fragments thereof. In another aspect, inhibition of SFEC with an antibody is useful for contraception.

Antibodies generated in accordance with the present invention may include, but are not limited to, polyclonal, monoclonal, chimeric (i.e “humanized” antibodies), single chain (recombinant), Fab fragments, and fragments produced by a Fab expression library. These antibodies can be used as diagnostic agents for the diagnosis of conditions or diseases characterized by in appropriate expression or overexpression of SFEC (including neoplastic disease), or in assays to monitor the effectiveness of an SFEC agonist, antagonist or inhibitor. The antibodies may be used with or without modification, and may be labeled by joining them, either covalently or non-covalently, with a reporter molecule. In addition, the antibodies can be formulated with standard carriers and optionally labeled to prepare therapeutic or diagnostic compositions.

Antibodies raised against SFEC can be generated using standard techniques, and include, but are not limited to, polyclonal, monoclonal, chimeric, single chain, Fab fragments, and Fab expression libraries. The antibodies generated can be formulated with standard carriers and optionally labeled to prepare therapeutic or diagnostic compositions. In one embodiment, a composition is provided comprising a SFEC specific antibody and a pharmaceutically acceptable carrier. In one embodiment the composition further comprises a surfactant, adjuvant, excipient or stabilizer. In general, water, saline, aqueous dextrose, and related sugar solution, and glycols such as, propylene glycol or polyethylene glycol, are the liquid carriers, particularly for injectable solutions.

Antibodies directed against SFEC peptides or derivative, homologs, or fragments thereof, may be generated using methods that are well known in the art. For instance, U.S. patent application Ser. No. 07/481,491, which is incorporated by reference herein in its entirety, discloses methods of raising antibodies to sperm-specific proteins. For the production of antibodies, various host animals, including but not limited to rabbits, mice, and rats, can be immunized by injection with an SFEC peptide or derivative, homolog, or fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. In one aspect, the SFEC peptide comprises SEQ ID NO:2. In another, the SFEC peptide comprises SEQ ID NO:4.

For the preparation of monoclonal antibodies, any technique which provides for the production of antibody molecules by continuous cell lines in culture may be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) may be employed to produce human monoclonal antibodies. In another embodiment, monoclonal antibodies are produced in germ-free animals utilizing the technology described in international application no. PCT/US90/02545, which is incorporated by reference herein in its entirety.

For preparation of monoclonal antibodies directed toward the sequence of SEQ ID NO: 2, SEQ ID NO: 4, or fragment thereof, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In one embodiment, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for egg surface proteins together with genes from a human antibody molecule of appropriate biological activity can be used; such “humanized” antibodies are within the scope of this invention.

In accordance with the invention, human antibodies may be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be employed; such antibodies are within the scope of the present invention. Once specific monoclonal antibodies have been developed, the preparation of mutants and variants thereof by conventional techniques is also available.

In one embodiment, techniques described for the production of single-chain antibodies (U.S. Pat. No. 4,946,778, incorporated by reference herein in its entirety) are adapted to produce protein-specific single-chain antibodies directed against an SFEC protein, or a derivative, homolog, or fragment thereof.

In another embodiment, the techniques described for the construction of Fab expression libraries (Huse et al., 1989, Science 246:1275-1281) are utilized to allow rapid and easy identification of monoclonal Fab fragments possessing the desired specificity for sperm-specific antigens, proteins, derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M113 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters. Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for derivatives of proteins. Derivatives can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The antibodies can be used in methods known in the art relating to the localization and activity of SFEC, e.g., for imaging these proteins, measuring levels thereof in appropriate physiological samples, in diagnostic methods, etc. The antibodies generated against SFEC antigens can also be used as contraceptive or sterilization agents (i.e. passive immunotherapy), or for use in diagnostic immunoassays or the generation of antiidiotypic antibodies. For example, in one embodiment SFEC antibodies are isolated (e.g., immunoaffinity chromatography, centrifugation, precipitation, etc.) and used in diagnostic immunoassays, or the antibodies may be used to monitor treatment and/or disease progression. Any immunoassay system known in the art, such as those listed supra, may be used for this purpose including but not limited to competitive and noncompetitive assay systems using techniques such as radioimmunoassays, ELISA (enzyme-linked immunosorbent assays), “sandwich” immunoassays, precipitin reactions, gel diffusion precipitin reactions, immunodiffusion assays, agglutination assays, complement-fixation assays, immunoradiometric assays, fluorescent immunoassays, protein A immunoassays and immunoelectrophoresis assays.

Another embodiment of the present invention is directed to small molecule inhibitors of SFEC and their use to decrease the motility of mammalian sperm and thus serve as a contraceptive agent. In one embodiment a method of contraception is provided wherein said method comprises the steps of inhibiting the activity of SFEC. Alternatively, the SFEC inhibitory composition may comprise an antisense or interference RNA that prevents or disrupts the expression or activity of SFEC in mammalian sperm cells. In accordance with one embodiment the fertility inhibiting composition comprises one or more active agents selected from the group consisting of small molecule inhibitors, antibodies, antisense RNA and interference nucleic acid sequences.

Interference RNA in mammalian systems requires the presence of short interfering RNA (siRNA), which consists of 19-22 nt double-stranded RNA molecules, or shRNA, which consists of 19-29 nt palindromic sequences connected by loop sequences. Down regulation of gene expression is achieved in a sequence-specific manner by pairing between homologous siRNA and target RNA. A system for the stable expression of siRNA or shRNA was utilized to generate transgenic animals (Hasuwa et al. FEBS Lett 532, 227-30 (2002), Rubinson et al. Nat Genet 33, 401-6 (2003) and Carmell et al. Nat Struct Biol 10, 91-2 (2003)) and can be used in accordance with the present invention to produce animals whose fertility can be regulated. A conditional interference RNA-based transgenic system would provide the additional benefit of being able to control the level of gene expression at any given stage during the life of the animal. Such a regulatable system would also have value in livestock and domesticated animals.

The invention provides a method of contraception, comprising inhibiting SFEC.

The invention provides a method of diagnosing a disease or disorder, particularly a fertility related disease or disorder, said method comprising measuring the expression, levels, or function of SFEC in a subject having a disease or disorder related to aberrant expression, levels or function of SFEC. In one aspect, the levels of SFEC in a test subject are compared to the levels of SFEC in a control subject or sample.

The invention also includes a kit comprising the composition of the invention and an instructional material which describes administering the composition to a sample. In another embodiment, this kit comprises a (preferably sterile) solvent suitable for dissolving or suspending the composition of the invention prior to administering the antibody.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the antibodies of the invention or be shipped together with a container which contains the antibody. Alternatively, the instructional material may be shipped separately from the container with the intention that the instructional material and the compound be used cooperatively by the recipient.

The invention also encompasses the use pharmaceutical compositions of an appropriate compound, analog, or derivative thereof to practice the methods of the invention, the composition comprising at least one appropriate compound, analog, or derivative thereof and a pharmaceutically-acceptable carrier.

As used herein, the term “pharmaceutically-acceptable carrier” means a chemical composition with which an appropriate compound may be combined and which, following the combination, can be used to administer the appropriate compound to a mammal. Preferably, the mammal is a human.

The pharmaceutical compositions useful for practicing the invention may be administered to deliver a dose of between 1 ng/kg/day and 100 mg/kg/day.

Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

Compounds which are identified using any of the methods described herein may be formulated and administered to a mammal for treatment of the diseases or disorders disclosed herein.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, parenteral, topical, pulmonary, intranasal, buccal, ophthalmic, intrathecal, intraurethral, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyamide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

The invention is now described with reference to the following examples. These examples are provided for the purpose of illustration only and the invention should in no way be construed as being limited to these examples but rather should be construed to encompass any and all variations which become evident as a result of the teaching provided herein.

In accordance with the present invention, as described above or as discussed in the Examples below, there can be employed conventional clinical, chemical, cellular, histochemical, biochemical, molecular biology, microbiology and recombinant DNA techniques which are known to those of skill in the art. Such techniques are explained fully in the literature.

The invention should not be construed to be limited solely to the assays and methods described herein, but should be construed to include other methods and assays as well. One of skill in the art will know that other assays and methods are available to perform the procedures described herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without wishing to be bound by any particular theory, it is hypothesized that the lack of mitochondria in the principal piece and limitations in diffusion of mitochondrial ATP from the sperm midpiece to the distal flagella implies the presence in the principal piece of independent mechanisms for energy production and transport. To identify distal flagellar proteins involved in energy production and transport, a proteomic approach was undertaken to microsequence the insoluble proteins comprising the human fibrous sheath.

### Example 1

Identification of SFEC

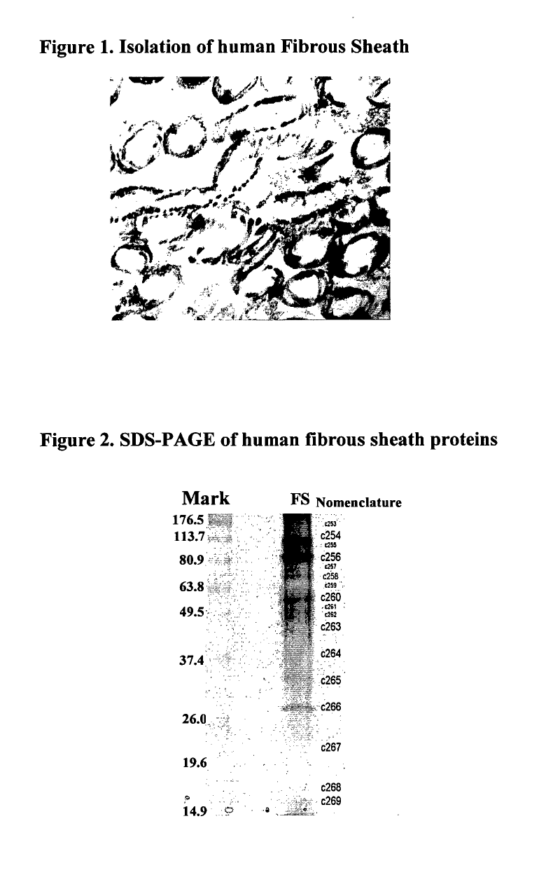

To further characterize the proteins that comprise the fibrous sheath, fibrous sheaths were isolated from human sperm using mechanical and biochemical dissection methods using techniques previously described (see Kim et al., Mol Hum Reprod., 1997, (4):307-13). Electron microscopic observations of the dissected fraction revealed a highly purified preparation consisting exclusively of fibrous sheath ribs and longitudinal columns (see FIG. 1). The fibrous sheath proteins were extracted and one dimensional SDS-PAGE was conducted (see Kim et al., Mol Hum Reprod. 1997, (4):307-13). 2-D gel analysis of the isolated fibrous sheath, using classical urea extraction methods, proved unsuccessful due to the insolubility of the fibrous sheath proteins in the Celis buffers employed in isoelectric focusing.

The results of one-dimensional SDS-PAGE reveal that the fibrous sheath contains at least 17 distinct Coomassie staining protein bands (FIG. 2). These bands were assigned a nomenclature of C253-C269, and each band was cored and microsequenced by tandem mass spectrometry. The results indicated that the isolated fibrous sheath preparation contained many proteins (see Table 1) that had been previously characterized as fibrous sheath components including roporrin, AKAP3, AKAP4, GST mu, and GAPDH-2. These findings confirmed the purity of the isolated fibrous sheath preparation. However, more significantly, microsequencing of isolated human fibrous sheath also revealed the presence of five glycolytic proteins, not previously reported to be associated with the fibrous sheath. These enzymes are aldolase A, sorbitol dehydrogenase, lactate dehydrogenase, triose phosphate isomerase (TPI), pyruvate kinase.

The addition of 5 new components to the 2 previously known glycolytic enzymes contained in the human fibrous sheath conclusively establishes glycolysis as a process occurring in the principal piece of the sperm flagellum, independent of ATP generation in the mitochondria. Glycolysis is an essential metabolic pathway that may proceed in the absence of oxygen to generate ATP. Accordingly, these findings demonstrate that the fibrous sheath is a flagellar sub-compartment for the glycolytic pathway to generate ATP under anaerobic condition.

The identification of sorbitol dehydrogenase, a key enzyme in polyol metabolism, strongly suggests a role for this pathway involving the conversion of sorbitol to fructose for use as an energy source in flagellar motility. Bioinformatic analyses of aldolase A, triose phosphate isomerase and pyruvate kinase peptides indicate these enzymes represent somatic forms, whereas purified fibrous sheath contained the testis specific isoform of lactate dehydrogenase, LDHC.

Most strikingly, a novel ADP/ATP translocase, named sperm flagella energy carrier (SFEC) herein, was identified in the fibrous sheath fraction. Provided herein is a description of the cloning this protein, expressing it as a recombinant protein, producing a specific polyclonal antibody against SFEC to help localize the protein, producing a nucleic acid probe to localize expression of SFEC, and localization of the protein. This localization establishes SFEC as a new member of the adenine nucleotide translocase (ANT) family distinct from ANTs that have been isolated from inner mitochondrial membranes. Northern analysis, dot blot analysis, as well as EST databases indicate that SFEC is a testis specific ANT. GenBank has provided accession number AY550240 for the human SFEC nucleic acid sequence (SEQ ID NO:1).

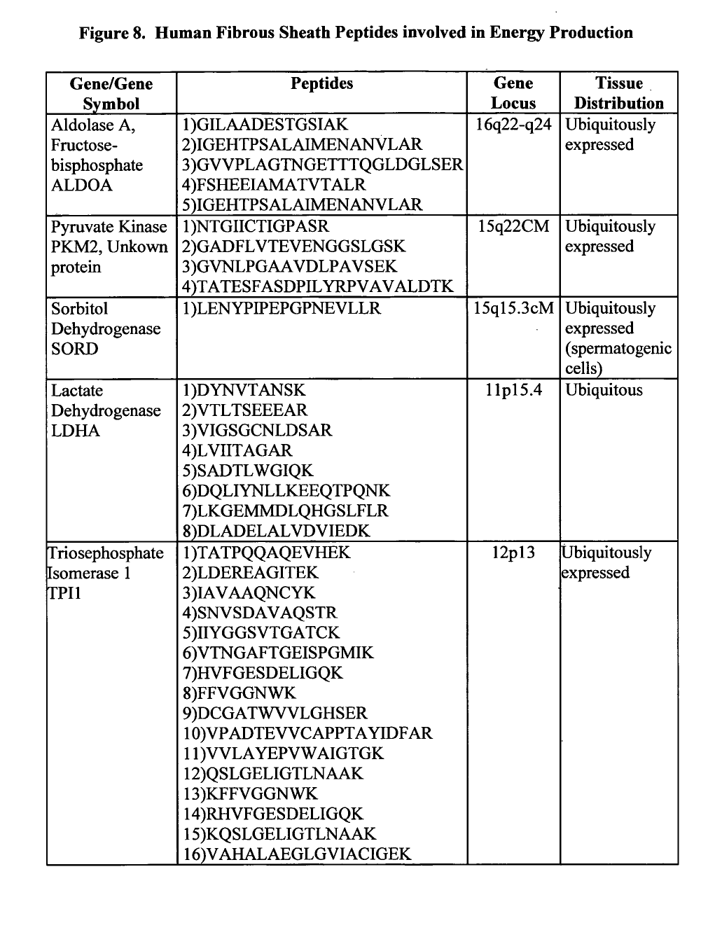

Bioinformatic analysis of the five glycolytic peptides that were obtained from the human fibrous sheath indicate that the glycolytic enzymes represent the somatic form of each enzyme (see FIG. 8), with the exception of the testis specific form of lactate dehydrogenase, LDHC. Although testis isoforms of triose phosphate isomerase have been identified in human (Strausberg et al., Proc. Natl. Acad. Sci. U.S.A. 99 (26), 16899-16903 (2002)), the peptides identified in the fibrous sheath represent the somatic form of TPI rather than the testis isoform. This indicates the fibrous sheath glycolytic machinery is comprised of two subsets of glycolytic enzymes: testis specific as well as somatic isoforms.

In addition, several new uncharacterized hypothetical proteins were identified as components of the fibrous sheath. These include the hypothetical protein FLJ23338 from band C253, hypothetical protein R30953_{—}1 from band C259 and hypothetical protein DKFZp434N1235 from band C265. The C265 band hypothetical protein DKFZp434N1235 has been cloned, sequenced, and further studied by bio-informatic analysis. Genes were annotated by the Ensembl automatic analysis pipeline using either a GeneWise model form a human/vertebrate protein, a set of aligned human cDNAs followed by GenomeWise for ORF prediction for from Genscan exons supported by protein, cDNA and EST evidence. GeneWise models are further combined with available aligned cDNAs to annotate UTRs. Bio-informatic comparison of the band C265 protein with other known proteins revealed the highest homology (with a 69% identity and 79% similarity) to the amino acid sequence of adenine nucleotide translocase 1, ANT1, in human heart/skeletal muscle, and a 67% identity and 80% similarity to ANT3 of human liver. C265 protein also revealed a 67% identity and 79% similarity with a human fibroblast isoform (ANT2). The human SFEC protein is 315 amino acids in length, has a molecular weight of 35021.78 daltons, an isoelectric point of 10.4632, a charge of 24.5 and an average residue weight of 111.180. The functional domains of human SFEC are indicated in FIG. 6.

The nucleotide sequence of the human SFEC mRNA covers 1727 bp (SEQ ID NO:1) including an open reading frame that yields a protein of 315 amino acid residues. SEQ ID NO:1 is as follows:

aagtgccactttctcgccagtacgatgctgcagcggttttccggttttccgcttcccttcatcgtagctcccgtact catttttagccactgctgccggtttttatatccttctccatcatgcatcgtgagcctgcgaaaaagaaggcagaaaagcggc tgtttgacgcctcatccttcgggaaggaccttctggccggcggagtcgcggcagctgtgtccaagacagcggtggcgcc catcgagcgggtgaagctgctgctgcaggtgcaggcgtcgtcgaagcagatcagccccgaggcgcggtacaaaggc atggtggactgcctggtgcggattcctcgcgagcagggtttcttcagttttggcgtggcaatttggcaaatgttattcggtat tttccaacacaagctctaaactttgcttttaaggacaaatacaagcagctattcatgtctggagttaataaagaaaaacagttc tggaggtggtttttggcaaacctggcttctggtggagctgctggggcaacatccttatgtgtagtatatcctctagattttgcc cgaacccgattaggtgtcgatattggaaaaggtcctgaggagcgacaattcaagggtttaggtgactgtattatgaaaata gcaaaatcagatggaattgctggtttataccaagggtttggtgtttcagtacagggcatcattgtgtaccgagcctcttatttt ggagcttatgacacagttaagggtttattaccaaagccaaagaaaactccatttcttgtctcctttttcattgctcaagttgtga ctacatgctctggaatactttcttatccctttgacacagttagaagacgtatgatgatgcagagtggtgaggctaaacggca atataaaggaaccttagactgctttgtgaagatataccaacatgaaggaatcagttccttttttcgtggcgccttctccaatgtt cttcgcggtacagggggtgctttggtgttggtattatatgataaaattaaagaattctttcatattgatattggtggtaggtaatc gggagagtaaattaagaaatacatggatttaacttgttaaacatacaaattacatagctgccatttgcatacattttgatagtgt tattgtctgtattttgttaaagtgctagttctgcaataaagcatacattttttcaagaatttaaatactaaaaatcagataaatgtg gattttcctcccacftagactcaaacacattttagtgtgatatttcatttattataggtagtatattttaattgttagtttaaaattctt tttatgattaaaaattaatcatataatcctagattaatgctgaaatctaggaaatgaaagtagcgtcttttaaattgctattcattt aatatacctgttttcccatcttttgaagtcatatggtatgacatatttcttaaaagcttatcaatagatgtcatcatatgtgtaggc agaaataagctttgttctatatctcttctaagacagttgttattactgtgtataatatttacagtatcagcctttgattatagatgtg atcatttaaaatttgataatgactttagtgacattataaaactgaaactggaaaataaaatggcttatctgctgatgtttatcttta aaataaataaaatcttgctagtgtgaatacaaaaaaaaaaaaaaaa.

The gene structure of SFEC spans approximately 43.8 kb divided into 6 exons and 5 introns. The human SFEC gene was localized to chromosome 4q28.2, while murine SFEC was localized to chromosome 3B. The other known human ADP/ATP carrier proteins in the same family such as heart/skeletal muscle IsoformT1 (ANT 1) and liver isoformT2 (ANT 3) were localized to chromosome 4 q35.1 and chromosome X p22.33, respectively.

Fibroblast isoform (ANT2) was localized to chromosome X q24. From this evidence indicating the presence of an uncharacterized unique gene the C265 protein is believed to be a novel member of the family of ADP/ATP Carrier Proteins, also known as the ADP/ATP Translocase, or alternatively, Adenine Nucleotide Translocator or ANT. Since the C265 protein was isolated from the fibrous sheath and because a role in signal transduction or glycolysis or both is likely, the novel protein has been designated as a sperm flagellar energy carrier protein or SFEC. At this time, it is not yet apparent if SFEC functions as an ATP reserve (e.g., storage/sink) or as an ATP carrier which shuttles ATP to the axoneme.

It is known that testis specific isoforms of hexokinase 1 (Hk1-sa, Hk1-sb and Hk1-sc) are produced from a single somatic gene Hk1 by alternative splicing. In contrast, the testis specific form of GAPDH, GAPDS, is encoded by a unique gene locus Gapds in mouse and GAPDH2 in humans. Thus, of the two known glycolytic enzymes localized in the flagellum, testis specific isoforms exist, and these are generated by either alternative splicing or expression of unique genes. However, it is very interesting that the bioinformatic analysis of the peptides isolated from the human fibrous sheath indicates that they are all somatic isoforms and do not represent testis specific isoforms, although such forms have been described for triose phosphate isomerase and LDHC, the germ cell-specific member of the lactate dehydrogenase family. This supports the fibrous sheath as being comprised of testis specific and somatic members of the glycolytic enzyme families.

The nucleic acid sequences of human and mouse SFEC are shown as SEQ ID NO:1 and SEQ ID NO:3, respectively, and the deduced human and mouse amino acid sequences are shown as SEQ ID NO:2 and SEQ ID NO:4, respectively. The human and mouse SFEC shared 83% identity and 89% similarity of protein sequences. The sequence for the 315 amino acid residue human SFEC is SEQ ID NO:2:

MetHisArgGluProAlaLysLysLysAlaGluLysArgLeuPheAspAlaSerSerPhe GlyLysAspLeuLeuAlaGlyGlyValAlaAlaAlaValSerLysThrAlaValAlaProIleGluArg ValLysLeuLeuLeuGlnValGlnAlaSerSerLysGlnIleSerProGluAlaArgTyrLysGlyMet ValAspCysLeuValArgIleProArgGluGlnGlyPhePheSerPheTrpArgGlyAsnLeuAlaAs nValIleArgTyrPheProThrGlnAlaLeuAsnPheAlaPheLysAspLysTyrLysGlnLeuPheM etSerGlyValAsnLysGluLysGlnPheTrpArgTrpPheLeuAlaAsnLeuAlaSerGlyGlyAla AlaGlyAlaThrSerLeuCysvalValTyrProLeuAspPheAlaArgThrArgLeuGlyValAsplle GlyLysOlyProGluGluArgGlnPheLysGlyLeuGlyAspCysIleMetLysIleAlaLysSerAsp GlyIleAlaGlyLeuTyrGlnGlyPheGlyValSerValGlnGlylleIleValTyrArgAlaSerTyrPhe GlyAlaTyrAspThrValLysGlyLeuLeuProLysProLysLysThrProPheLeuValSerPhePhe IleAlaGlnValValThrThrCysSerGlylleLeuSerTyrProPheAspThrValArgArgArgMet MetMetGlnSerGlyGluAlaLysArgGlnTyrLysGlyThrLeuAspCysPheValLysIleTyrGl nHisGluGlylleSerSerPhePheArgGlyAlaPheSerAsnValLeuArgGlyThrGlyGlyAlaLe uValLeuValLeuTyrAspLysIleLysGluPhePheHisIleAspIleGlyGlyArg.

The mouse 1577 base nucleic acid sequence for SFEC is SEQ ID NO:3:

agtgtcgcttgagtgttggtgtggcctgcaggtgtccggttgcccggtcctctgtccaacatgtcgaacgaatc ctccaagaagcagtcttcaaagaaggcgctgttcgatccggtgtctttctcgaaggacctgctggccggcggggtcgcg gccgcggtgtcgaagacaactgtggcgcccatcgagcgtgtgaagctgctgctgcaggtgcaggcgtcctccaagca gataagccctgaggcgcgctacaagggcatgctggactgcctggtgcgcattcctcgtgagcaaggatttttaagttattg gcgtggcaatttggcaaatgttattcgatactttccaacacaagccttaaacttcgcttttaaggacaaatacaaagaactttt catgtctggtgttaataaagaaaaacagttctggagatggtttctagcaaacctggcttctggaggggctgctggagcaac atccttgtgtgtagtatacccactagattttgccagaacccgattaggtgttgatattggaaaaggtcctgagcagcggcag ttcacgggtttgggtgactgcattatgaaaatagccaagtcagatggactgattggtctataccaagggtttggtgtctctgtt cagggtatcattgtttaccgagcctcttactttggagcttatgacaccgttaagggcttattgccaaagccaaaggaaaccc catttcttgtctcttttatcattgctcaaatcgtgactacctgttctggaatactctcctatccctttgacacagttagaagacgta tgatgatgcagagtggggaatctgatcggcaatataaaggaaccatagactgctttctgaaaatctaccgtcatgaagga gttcctgccttcttccgtggtgccttctccaacatccttcgtggcacagggggtgctttggtcttggtgttatatgataaaatca aagagttcctcaacattgatgttggaggtagttcatcaggagattaaattgagaaatgcatatttctaatgtaaaaacatgaa aattacatagctgccatttttatatattttgatagtgtgttactactgtcagtgtctcttacagtatttgttctgcaataaagaaaag atttttttttcaagattttagtattaaaagtcaggacaaaaatttttttcacttagacccagaatcatatattaaggcattttattata ggtagtgtatgttacttctgttaaaaaattcttacacttgtgatgaacaccatataatgtgaaatatgaggaagtgtctttaaact tcaatttgcttagtacaacagtaatcccatcttttaggaattgtattgtatgaccaatagttgaaaagttgataatgacttagtga cactatcaaactatttgaaaagtataggttgggctattgctaatgtttagtcttgctagtgtatataaatctttgaacaagaaat ctctggacattagattttgtattctgtatcaataataaagcaagctcaaaactaaaaaaaaaaaaaaaaaaaaaaaaaaaaa a.

The mouse 320 amino acid residue SFEC protein described herein has the sequence of SEQ ID NO:4:

MetSerAsnGluSerSerLysLysGlnSerSerLysLysAlaLeuPheAspProValSerPhe SerLysAspLeuLeuAlaGlyGlyValAlaAlaAlaValSerLysThrThrValAlaProIleGluArg ValLysLeuLeuLeuGlnValGlnAlaSerSerLysGlnIleSerProGluAlaArgTyrLysGlyMet LeuAspCysLeuValArgIleProArgGluGlnGlyPheLeuSerTyrTrpArgGlyAsnLeuAlaAs nValIleArgTyrPheProThrGlnAlaLeuAsnPheAlaPheLysAspLysTyrLysGluLeuPheM etSerGlyValAsnLysGluLysGlnPheTrpArgTrpPheLeuAlaAsnLeuAlaSerGlyOlyAla AlaGlyAlaThrSerLeuCysvalValTyrProLeuAspPheAlaArgThrArgLeuGlyValAsplle GlyLysGlyProGluGlnArgGlnPheThrGlyLeuGlyAspCysIleMetLysIleAlaLysSerAsp GlyLeuIleGlyLeuTyrGlnGlyPheGlyValSerValGlnGlylleIleValTyrArgAlaSerTyr PheGlyAlaTyrAspThrValLysGlyLeuLeuProLysProLysGluThrProPheLeuValSerPhe IleIleAlaGlnIleValThrThrCysSerGlylleLeuSerTyrProPheAspThrValArgArgArgMet MetMetGlnSerGlyGluSerAspArgGlnTyrLysGlyThrIleAspCysPheLeuLysIleTyrArg HisGluGlyValProAlaPhePheArgGlyAlaPheSerAsnIleLeuArgGlyThrGlyGlyAlaLeu ValLeuValLeuTyrAspLysIleLysGluPheLeuAsnIleAspValGlyGlySerSerSerGlyAsp.

Microsequencing of Fibrous Sheath Proteins

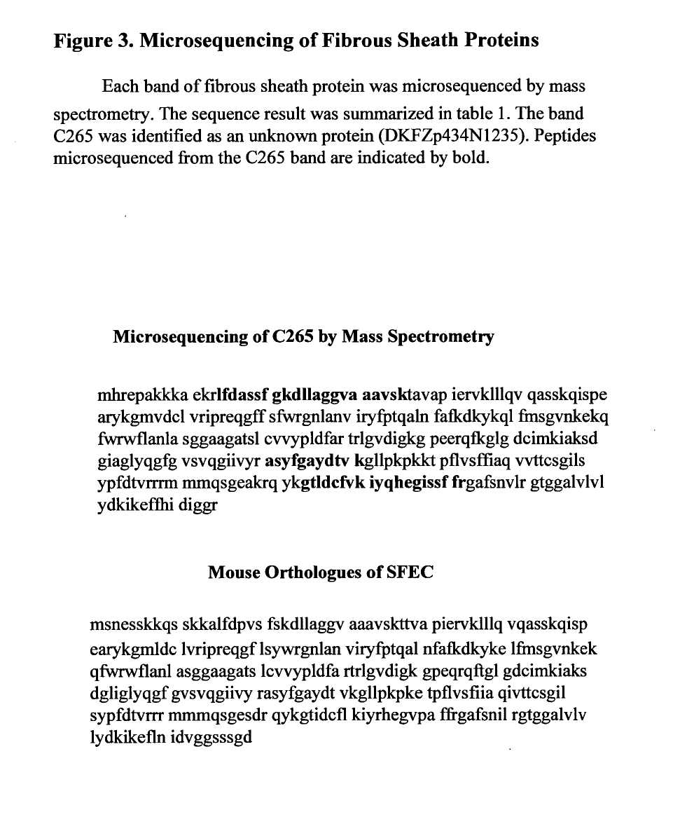

Each band of fibrous sheath protein which was identified electrophoretically was microsequenced by mass spectrometry. The sequence result is summarized in Table 1. The band C265 was identified as an unknown protein (DKFZp434N1235). Peptides microsequenced from the C265 band are indicated by bold-face type in FIG. 3.

Bioinformatics of C265

Genes were annotated by the Ensembl automatic analysis pipeline using either a GeneWise model from a human/vertebrate protein, a set of aligned human cDNAs followed by GenomeWise for ORF prediction or from Genscan exons supported by protein, cDNA and EST evidence. GeneWise models are further combined with available aligned cDNAs to annotate UTRs. The C265 was identified as a protein family of ADP ATP CARRIER ADP/ATP TRANSLOCASE ADENINE NUCLEOTIDE TRANSLOCATOR ANT

The nucleotide sequence of the SFEC mRNA covers 1727 bp including an open reading frame that yields a protein of 315 amino acid residues. The gene structure of SFEC spans approximately 43.8 kb divided into 6 exons and 5 introns. The human SFEC gene was localized to chromosome 4q28.2, while murine SFEC was localized to chromosome 3B.

SFEC Peptide Characteristics
Isoelectric point=10.4632
Charge=24.5
Molecular weight=35021.78
Number of residues=315
Ave. residue weight=111.180

Tissue-Specific Expression of SFEC

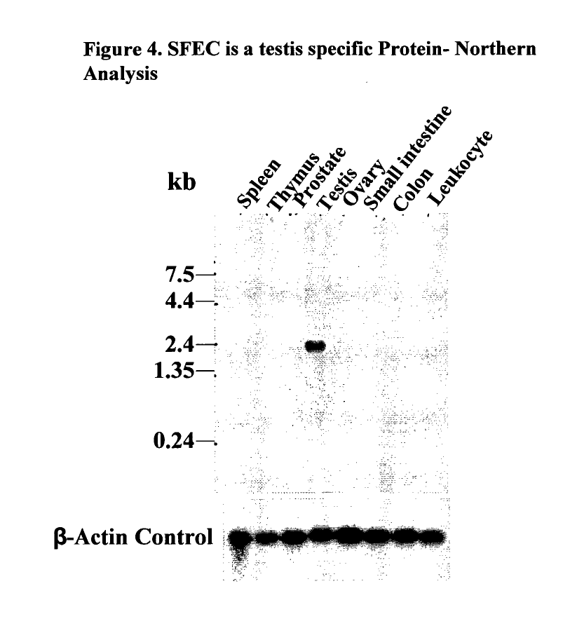

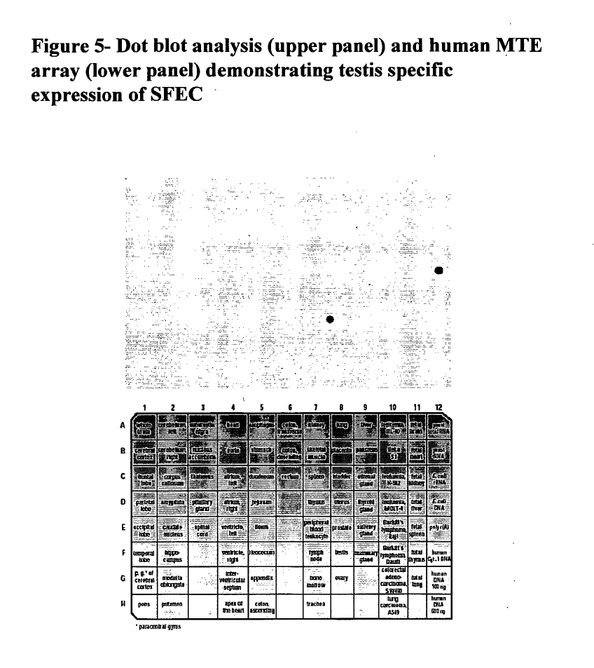

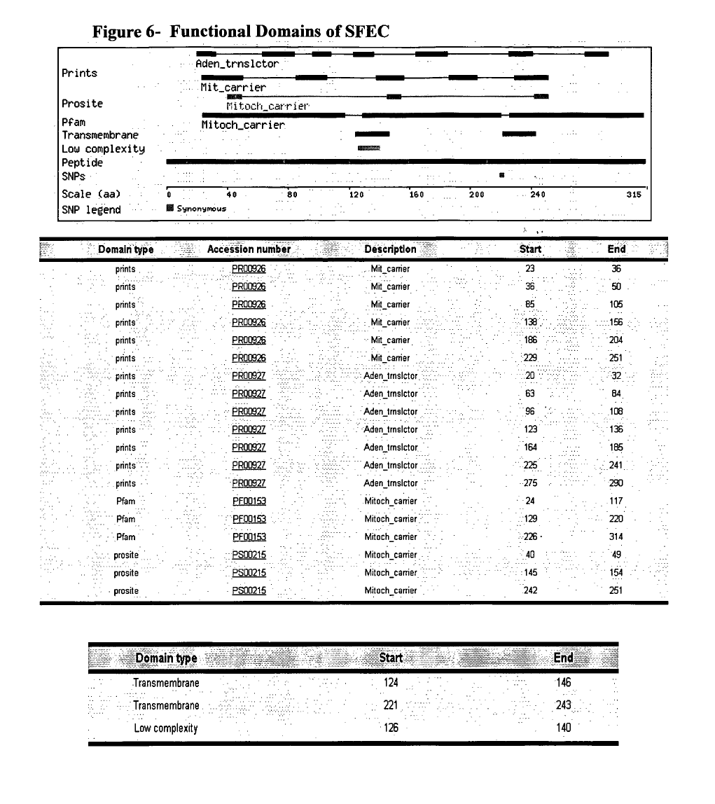

A series of Northern blot analyses, dot blot analyses, and MTE array determinations were performed (FIGS. 4 and 5). The data demonstrate that SFEC is expressed in testis, but not in the other tissues or cell types tested.

Functional Domains of SFEC

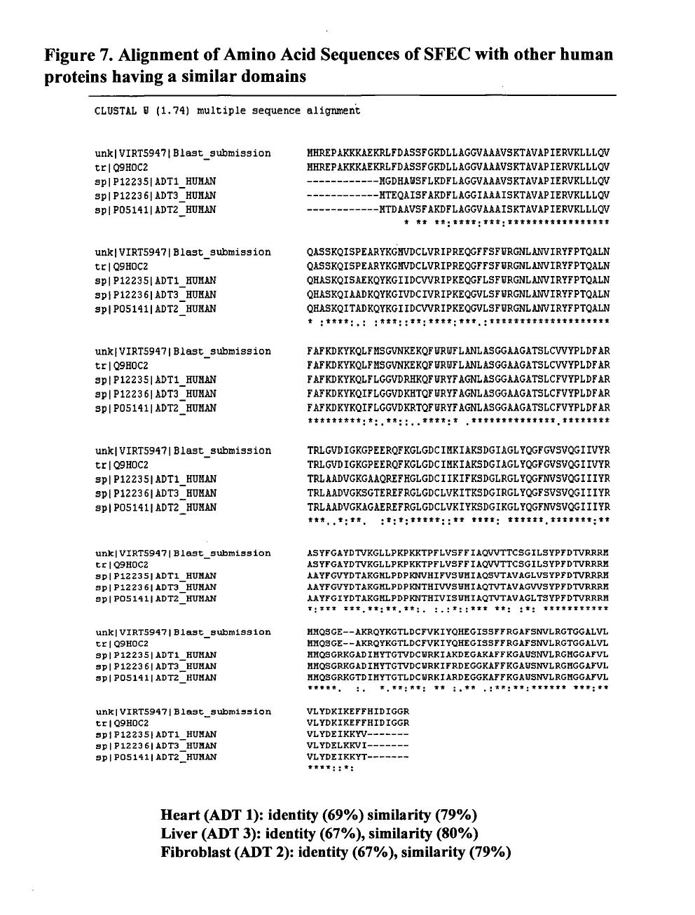

SFEC (315 amino acid residues) contains several functional domains such as mitochondrial carrier protein, mitochondrial substrate carrier, adenine nucleotide translocator 1 and two transmembrane domains (FIG. 6). The SFEC had a 69% identity and 79% similarity to amino acids of ADT1 human heart and a 67% identity and 80% similarity to ADT3 of human liver. Alignment of Amino Acid Sequences of SFEC with these proteins is shown in FIG. 7.

A summary of human fibrous sheath peptides known to be involved in energy production is provided in FIG. 8.

TABLE 1
Microsequencing Information on Isolated Fibrous Sheath Protein

| | | Protein NCBI#, |
|---|---|---|
| Band# (kDa) | Peptides microsequenced | MW (kDa) |
| C253 (140) | AKAP4 | 14759733, 89.6 |
| | AKAP3 | 14194457, 94.7 |
| | HSP90B | 668630, 83.3 |
| | Hypothetical FLJ23338 | 13375909, 82.4 |
| C254 (110) | AKAP4 | 14759733, 89.6 + |
| | | 14759737, 93.4 |
| | AKAP3 | 14194457, 94.7 + |
| | | 5454076, 94.8 |
| C255 (93) | AKAP3 | 14194457, 94.7 |
| | AKAP4 | 14759733, 89.6 + |
| | | 14759737, 93.4 |
| | HSP90B | 6680307, 83.7 |
| | HSP90A | 123678, 84.7 |
| C256 (84) | AKAP4 | 14759733, 89.6 + |
| | | 14759737, 93.4 |
| C257 (72) | AKAP4 | 14759733, 89.6 + |
| | | 14759737, 93.4 |
| | HSP70.2 | 13650446, 70.0 |
| | AKAP3 | 14194457, 94.7 |
| | HSP90B | 6680307, 83.3 |
| C258 | No protein (large amounts of | |
| | detergent only) | |
| C259 (59) | AKAP4 | 14759733, 89.6 + |
| | | 14759737, 93.4 |
| | Pyruvate Kinase | 4505839, 57.9 |
| | GAPDH-2 | 7657116, 44.5 |
| | Alpha Tubulin | 5174477, 50.2 |
| | Hypothetical R30953_1 | 10257429, 50.3 |
| C260 (57) | GAPDH-2 | 7657116, 44.5 |
| | AKAP4 | 14759733, 89.6 + |
| | | 14759737, 93.4 |
| | Alpha Tubulin | 5174477, 50.2 |
| | Beta Tubulin | 7106439, 49.7 |
| C261 (52) | Translation Elongation Factor 1a1 | 4503473, 42.8 |
| | Aldolase A | 4557305, 39.4 |
| C262 (49) | CGI-49 | 14731915, 47.2 |
| | Actin | 4501883, 42.0 |
| | Aldolase A | 4557305, 39.4 |
| | Tumor Necrosis Factor Type 1 AP | 14778059, 80.1 |
| | AKAP3 | 14194457, 94.7 |
| C263 (40) | AKAP4 | 14759733, 89.6 |
| | Sorbitol Dehydrogenase | 1583520, 38.3 |
| | Aldolase A | 4557305, 39.4 |
| C264 (38) | Lactate Dehydrogenase C | 5031857, 36.7 + |
| | | 4504973, 36.3 |
| | GAPDH | 7669492, 36.4 |
| C265 (32) | Triosephosphate Isomerase | 4507645, 26.7 |
| | Hypothetical DKFZp434N1235 | 113464, 32.9 |
| | GAPDH-2 | 7657116, 44.5 |
| | HSP70.2 | 13650446, 70.7 |
| | Casein aS1 | 115646, 24.5 |
| | ADP/ATP Carrier Protein | 113464, 32.9 |
| C266 (28.5) | Glutathione S-transferase Mu | 4504177, 26.7 |
| | GAPDH-2 | 7657116, 44.5 |
| C267 (24) | AKAP-binding protein(ropporin) | 13487902, 24.0 |
| | AKAP4 | 14759733, 89.6 |
| | Transcription Factor IIB | 8392875 |
| | RAB2 | 106185, 23.6 |
| C268 (18) | CAP-18 | 1706745, 19.3 |
| | SPANX-C | 13435137, 11.0 |
| C269 (15.5) | CAP-18 | 1706745, 19.3 |
| | HE3 Beta | 11641279, 17.6 |
| | Ribosomal Protein L22 | 4506613, 14.8 |

### Example 2

Expression and Purification of Recombinant SFEC Protein

A truncated construct (amino acid residues 4-120) of human SFEC (SEQ ID NO:2; 315 amino acid residues) was expressed in bacteria in order to raise a polyclonal antibody. Previous efforts to express the entire SFEC open reading frame were not successful in bacteria, presumably because of the existence of a putative transmembrane domain in the C-terminus. Gene specific primers were designed to create an Nco1 site at the 5′ end and a Not1 site at the 3′ end of the polymerase chain reaction (PCR) product according to the human SFEC cDNAs sequences. Primers (Forward primer: 5′-CATGCCATGGAGCCTGCGAAAAAGAAGGCAGAAAAG-3′ [SEQ ID NO:5] and Reverse primer: 5′-ATAGTTTAGCGGCCGCCTGTTTTTCTTTATTAACTCCAGA-3′ [SEQ ID NO:6]) were obtained from GIBCO BRL (Life Technologies, CA).

SFEC sequences also provided herewith are human nucleic acid (SEQ ID NO:1), human amino acid (SEQ ID NO:2), murine nucleic acid (SEQ ID NO:3) and murine amino acid (SEQ ID NO:4.

PCR was performed with 10 ng of human SFEC cDNAs as a template to obtain the truncated SFEC cDNA using a program of one 2 minutes cycle at 94° C. followed by 35 cycles of denaturation, annealing, and elongation at 94° C. for 30 second, 50° C. for 1 minute and 68° C. for 2 minutes. A product of 351 residues in length, which begins at residue position number 129 and ends at residue position number 479 of nucleotide sequences of human SFEC, were separated on a 1% NuSieve (FMC BioProducts, Rockland, Me.) agarose gel and sequenced in both directions using vector-derived and insert-specific primers to confirm the sequences.

The cDNA corresponding to the N-terminal 117 amino acids was cloned into the bacterial expression vector pET28b and transformed into Escherichia coli strain BLR (DE3) (Novagen, Madison, Wis.). A single colony was picked from a transformation plate to inoculate 2 liters of LB medium containing 50 μg/ml of Kanamycin and grown at 37° C. until the A₆₀₀ reached 0.5. The recombinant protein expression was induced at 37° C. for 3 hours with 1 mM IPTG (isopropyl-1-thio-β-D-galactopyranoside). The cells were centrifuged at 5,000 g for 15 minutes and suspended in BugBuster Protein Extraction reagent (Novagen, Madison, Wis.) containing rLysozyme (1 KU/ml) and Benzonase (25 units/ml) for the gentle disruption of the cell wall and degradation of DNA and RNA of the E. coli. The cells were centrifuged at 5,000 g for 15 minutes and the pellet of inclusion body was resuspended in 1× binding buffer (5 mM imidazole, 0.5 M NaCl, 20 mM Tris-HCl, pH 7.9) containing 100 μg/ml lysozyme and 8M urea.

The supernatant, urea soluble fraction, was loaded onto a Ni²⁺-activated His-binding resin (Novagen, Madison, Wis.) following the manufacturer's protocol. The recombinant protein was eluted with 400 mM imidazole in 1× binding buffer containing 8M urea. The eluted proteins were dialyzed in distilled water, lyophilized at −70° C. and performed further purification to a single band using a model 491 Prep Cell (Bio Rad). The purity of the isolated recombinant protein was confirmed by Coomassie and SYPRO Ruby stain (Bio-Rad).

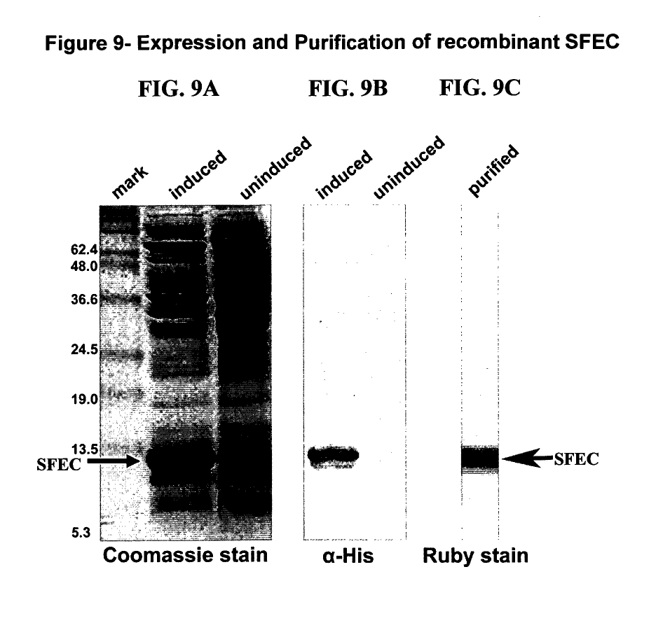

The recombinant SFEC protein containing six residues of histidine on the C-terminus of the protein was induced by 1 mM IPTG (FIG. 9A) and confirmed the protein expression using anti-histidine antibody (FIG. 9B). The purity of isolated protein of ˜13 kDa was verified by SYPRO Ruby stain (FIG. 9C).

### Example 3

Generation of a Rat Anti-Human SFEC Antibody

Approximately 100 μg of purified recSFEC protein in PBS emulsified with equal volume of Freund's complete adjuvant was injected subcutaneously and intramuscularly into each female Sprague Dawley rat. Animals were boosted two times at an interval of 21 days with 50 μg of recombinant protein in incomplete Freund's adjuvant and serum was collected 7 days after the second boost. Rats were sacrificed after confirmation of antibody production by Western blot analysis on the recombinant SFEC, human sperm, and isolated FS proteins.

Western Analyses of Anti-SFEC Antibody on the Human Sperm, Isolated FS and Recombinant Proteins

The reaction of SFEC antibody was tested by Western blot analysis on the recombinant SFEC, human sperm, and isolated FS proteins. The human swim up sperm proteins were extracted as previously described (Shetty et al., Biol. Reprod. 61(1):61-9 (1999)). Sperm were solubilized by constant shaking for 2 hours at 4° C. in a CELIS lysis buffer containing 9.8 M urea, 2% NP-40, 100 mM DTT and the protease inhibitors: 2 mM PMSF, 5 mM iodoacetamide, 5 mM EDTA, 3 mg/ml L-1-chlor-3-(4-tosylamido)-7-amino-2-heptanon-hydrochloride, 1.46 mM pepstatin A, and 2.1 mM leupeptin. Insoluble material was removed by centrifugation at 10000×g for 5 minutes, and the supernatant containing solubilized human sperm protein was subjected to one-dimensional electrophoresis.

The proteins resolved by one-dimensional SDS-PAGE were transferred onto nitrocellulose membranes and detected by the anti-SFEC antibody. The excess protein-binding sites on the membrane were blocked with PBS containing 5% (w/v) non-fat milk powder and 0.2% (w/v) Tween 20 (Merck-Schuchardt, Hohenbrunn, Germany) for 1 hour. The membrane was probed overnight at 4° C. with a rat polyclonal antiserum raised against SFEC protein. Anti-SFEC antibody was diluted 1:2000 with blocking solution. Preimmune sera were diluted same as post immune sera for control experiments. The membrane was then incubated for 45 minutes with an anti-rat immunoglobulin IgG-secondary antibody linked to horseradish peroxidase (Jackson Immuno Research Lab., West Grove, Pa. USA) diluted 1:5000 in blocking solution. The blot was developed with a chemiluminescent substrate (Pierce, Rockford, Ill.) or 3,3′,5,5′-tetramethylbenzidine (TMB) substrate solution (Kirkegaard and Perry Lab., Gaithersburg, Md. USA).

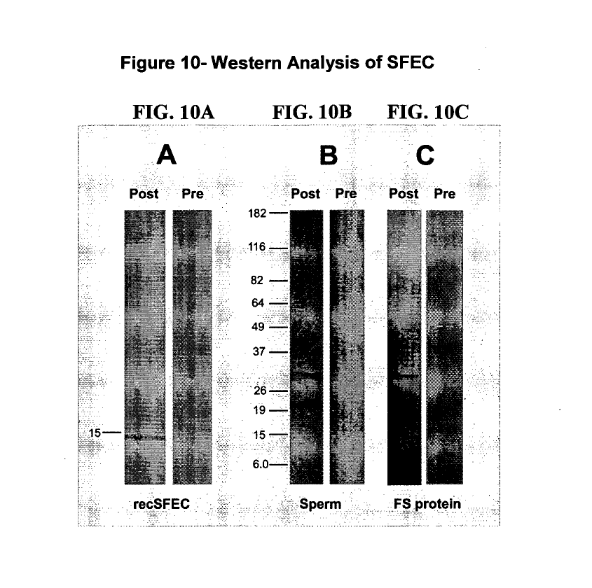

The positive signal elicited by anti-SFEC antibody was detected intensively on the 38, 32, 20 kDa of human sperm, but only 32 kDa on the isolated FS proteins (FIG. 10A). The human sperm and FS proteins were not detected by corresponding preimmune serum (FIG. 10B). This result demonstrates that a rat anti-human SFEC antibody recognizes SFEC as an immunogen, as well as human sperm, including FS proteins, indicating that SFEC is a component of FS proteins.

### Example 4

Localization of SFEC to the Principal Piece in Association with the FS of the Flagellum in Human Sperm

Swim-up human sperm were washed in PBS containing 0.2 mM PMSF, diluted to a concentration of a 1×10⁶ sperm/ml, and then spotted onto glass slides. The sperm were air-dried, and fixed with 4% paraformaldehyde for 30 minutes at room temperature. After washing 3 times in PBS, the samples were blocked in 10% normal goat serum in PBS overnight at 4° C. The sperm were then incubated with 1:50 dilution of the rat anti-recombinant SFEC antibody or its pre-immune serum in the blocking solution for 2 hours at room temperature. The slides were then washed 3 times×5 minutes in PBS, and the secondary antibody, goat anti-rat IgG FITC conjugated (Jackson ImmunoResearch), were applied at 1:100 dilution in 10% normal goat serum in PBS for 1 hour at room temperature. The slides were washed 3 times, 5 minutes/wash, in PBS. The Slow Fade-Light Antifade Kit containing DAPI (Molecular Probes, Inc.) was used to stain DNA of the sperm and to reduce the fading rate of the fluorescein.

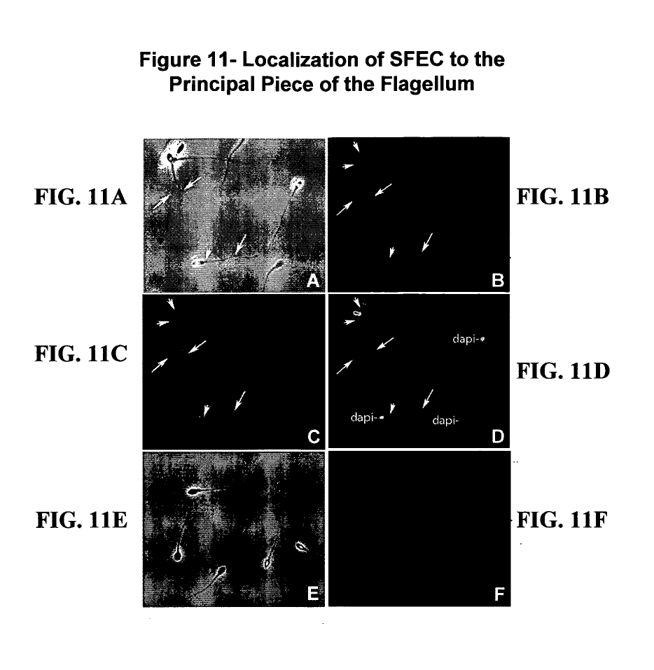

Indirect immunofluorescence analysis of human swim-up sperm using rat serum against recombinant human SFEC protein localized to the entire principal piece of the flagellum with no staining in the midpiece, endpiece or in the head (FIG. 11 B, C, D). Pre-immune serum showed no immunofluorescence in human sperm (FIG. 11 F). Interestingly only approximately 50% of sperm were recognized by the SFEC antibody which is directed against N-terminal 117 amino acids. This result is supported by a previous report that the accessibility of the N-terminal residues depends on the conformational state of the ADP/ATP carrier (Brandolin et al., Biochemistry 28:1093-1100 (1989)). This result indicates that each spermatozoon has different conformation states related to function for ADP/ATP translocation in the principal piece of the flagellum.

In summary, the present disclosure provides that SFEC is a novel and unique protein located in the principal piece of the sperm tail and is an appropriate target for a contraceptive drug. This proteomic analysis: 1) expands an understanding of the complement of enzymes involved in energy production and translocation in the principal piece; 2) supports a role for the fibrous sheath in flagellar glycolysis as well as polyol metabolism; 3) indicates that the glycolytic machinery within the principal piece includes somatic as well as testis specific isoenzymes; and 4) provides support for distal flagellar energy metabolism occurring independently from the midpiece mitochondrial sheath. Most importantly, a new hypothesis is advanced that a testis specific ATP/ADP carrier, SFEC, mediates ATP translocation to dynein ATPases involved in sperm motility, defining SFEC as a new contraceptive drug target, and providing a link between energy production and transport in the distal flagella.

Other methods which were used but not described herein are well known and within the competence of one of ordinary skill in the art of biochemistry, cell biology, and molecular biology.

The disclosures of each and every patent, patent application, and publication cited herein are hereby incorporated herein by reference in their entirety. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

While this invention has been disclosed with reference to specific embodiments, it is apparent that other embodiments and variations of this invention may be devised by others skilled in the art without departing from the true spirit and scope of the invention. The appended claims should be construed to include all such embodiments and equivalent variations.

What is claimed is:

1. A mammalian sperm flagellar energy carrier protein, or a homolog, derivative, or fragment thereof, wherein said protein comprises the amino acid sequence SEQ ID NO:2 or SEQ ID NO:4, or a homolog, derivative, or fragment thereof.

2. The sperm flagellar energy carrier protein of claim 1, wherein said protein is localized to the principal piece of the sperm flagellum.

3. The sperm flagellar energy carrier protein of claim 1, wherein said protein comprises the amino acid sequence SEQ ID NO:2 or an amino acid sequence substantially similar to SEQ ID NO:2.

4. The sperm flagellar energy carrier protein of claim 1, wherein said protein is an adenine nucleotide translocase.

5. A pharmaceutical composition comprising the sperm flagellar energy carrier protein of claim 1 and a pharmaceutically-acceptable carrier.

6. An isolated nucleic acid comprising a nucleic acid sequence, or a homolog, derivative, or fragment thereof, encoding a sperm flagellar energy carrier protein, or a homolog, derivative, or fragment thereof.

7. A vector comprising the isolated nucleic acid of claim 6.

8. The vector of claim 7, said vector further comprising a nucleic acid specifying a promoter/regulatory sequence operably linked thereto.

9. A host cell comprising the vector of claim 7.

10. The host cell of claim 9, wherein said cell is a mammalian cell.

11. A host cell comprising the vector of claim 8.

12. The host cell of claim 11, wherein said cell is a mammalian cell.

13. The isolated nucleic acid of claim 6, wherein said isolated nucleic acid comprises a nucleic acid having the sequence SEQ ID NO: 1 or NO:3, or a sequence substantially similar to SEQ ID NO: 1 or SEQ ID NO:3.

14. The isolated nucleic acid of claim 6, wherein said isolated nucleic comprises a nucleic acid sequence encoding an SFEC protein comprising SEQ ID NO:2 or SEQ ID NO:4.

15. A composition comprising an isolated nucleic acid complementary to an isolated nucleic acid encoding a sperm flagellar energy carrier protein, or a homolog, derivative, or fragment thereof, and a pharmaceutically-acceptable carrier.

16. An antibody that specifically binds to a sperm flagellar energy carrier protein, or a homolog, derivative, or fragment thereof.

17. The antibody of claim 6, wherein the antibody specifically binds to a sperm flagellar energy carrier protein comprising the amino acid sequence SEQ ID NO: 2, or a homolog, derivative, or fragment thereof.

18. The antibody of claim 17, wherein said antibody is a monoclonal antibody.

19. A pharmaceutical composition comprising the antibody of claim 16 and a pharmaceutically acceptable carrier.

20. An antigenic composition comprising a protein having the amino acid sequence SEQ ID NO: 2, or an antigenic homolog, derivative, or fragment thereof, and a pharmaceutically-acceptable carrier.

21. The composition of claim 20, further comprising an adjuvant.

22. A method of diagnosing a sperm flagellar energy carrier protein-associated disease or disorder related to aberrant sperm flagellar energy carrier protein expression, function, or levels, said method comprising obtaining a sample from a subject, measuring sperm flagellar energy carrier protein expression, function, or levels in said sample, wherein different amounts of sperm flagellar energy carrier protein expression, function, or levels in the sample relative to the amounts of sperm flagellar energy carrier protein expression, function, or levels in a sample from a control subject not having the sperm flagellar energy carrier protein-associated disease or disorder, indicates the presence of the sperm flagellar energy carrier protein-associated disease or disorder.

23. A method of treating a sperm flagellar energy carrier protein-associated disease or disorder in a subject in need of such treatment, comprising administering to the subject an effective amount of a composition comprising at least one isolated nucleic acid comprising a nucleic acid sequence encoding a sperm flagellar energy carrier protein, or a biologically active homolog, derivative, or fragment of said sperm flagellar energy carrier protein, and a pharmaceutically-acceptable carrier.

24. A method of treating a sperm flagellar energy carrier protein-associated disease or disorder in a subject in need of such treatment, comprising administering to the subject an effective amount of a composition comprising at least one regulator of sperm flagellar energy carrier protein expression, function, or levels, and a pharmaceutically-acceptable carrier.

25. A method of regulating sperm flagellar energy carrier protein expression, function, or levels in a subject, said method comprising administering to the subject an effective amount of a composition comprising at least one regulator of sperm flagellar energy carrier protein expression, function, or levels, and a pharmaceutically-acceptable carrier.

26. The method of claim 25, wherein said regulator of sperm flagellar energy carrier protein expression, function, or levels is an inhibitor of sperm flagellar energy carrier protein expression, function, or levels.

27. The method of claim 26, wherein said regulator is an antibody.

28. A kit for treating a sperm flagellar energy carrier protein-associated disease or disorder in a subject, said kit comprising a sperm flagellar energy carrier protein expression, function, or level regulating amount of a composition comprising a regulator of sperm flagellar energy carrier protein, and a pharmaceutically-acceptable carrier, said kit further comprising an applicator, and an instructional material for the use thereof.

29. A kit for regulating sperm flagellar energy carrier protein expression, function or levels, said kit comprising a sperm flagellar energy carrier protein regulating amount of a composition comprising a regulator of sperm flagellar energy carrier protein expression, function, or levels and a pharmaceutically-acceptable carrier, said kit further comprising an applicator, and an instructional material for the use thereof.

30. The kit of claim 29, wherein said regulator of sperm flagellar energy carrier protein expression, function, or levels inhibits sperm flagellar energy carrier protein expression, function, or levels.

31. The kit of claim 30, wherein said regulator is an antibody.

32. A method of identifying an inhibitor of sperm flagellar energy carrier protein expression, function, or levels in a cell, said method comprising contacting a cell with a test compound, measuring sperm flagellar energy carrier protein expression, function, or levels in said cell, wherein lower levels of sperm flagellar energy carrier protein expression, function, or levels in said cell, compared with sperm flagellar energy carrier protein expression, function, or levels in an otherwise identical cell not contacted with said test compound, is an indication that said test compound is an inhibitor of sperm flagellar energy carrier protein expression, function, or levels in a cell.

33. The method of claim 32, wherein said cell is a human cell.

34. The method of claim 32, wherein said inhibitor is an antibody.

35. The method of claim 34, wherein said antibody is directed against a sperm flagellar energy carrier protein, or a homolog, derivative, or fragment thereof, having the sequence SEQ ID NO:2, or a sequence substantially similar to SEQ ID NO:2.

36. The method of claim 32, wherein said method identifies a compound which inhibits sperm flagellar energy carrier protein adenine nucleotide translocase function.

37. A compound identified by the method of claim 32.


SEQUENCE LISTING (4 sequences)

SEQ ID NO: 1 - DNA, 1727 bp, homo sapiens
aagtgccactttctcgccagtacgatgctgcagcggttttccggttttccgcttcccttcatcgtagctcccgtactcatttttagccactgctgccggtttttatatccttctccatcatgcatcgtgagcctgcgaaaaagaaggcagaaaagcggctgtttgacgcctcatccttcgggaaggaccttctggccggcggagtcgcggcagctgtgtccaagacagcggtggcgcccatcgagcgggtgaagctgctgctgcaggtgcaggcgtcgtcgaagcagatcagccccgaggcgcggtacaaaggcatggtggactgcctggtgcggattcctcgcgagcagggtttcttcagtttttggcgtggcaatttggcaaatgttattcggtattttccaacacaagctctaaactttgcttttaaggacaaatacaagcagctattcatgtctggagttaataaagaaaaacagttctggaggtggtttttggcaaacctggcttctggtggagctgctggggcaacatccttatgtgtagtatatcctctagattttgcccgaacccgattaggtgtcgatattggaaaaggtcctgaggagcgacaattcaagggtttaggtgactgtattatgaaaatagcaaaatcagatggaattgctggtttataccaagggtttggtgtttcagtacagggcatcattgtgtaccgagcctcttattttggagcttatgacacagttaagggtttattaccaaagccaaagaaaactccatttcttgtctcctttttcattgctcaagttgtgactacatgctctggaatactttcttatccctttgacacagttagaagacgtatgatgatgcagagtggtgaggctaaacggcaatataaaggaaccttagactgctttgtgaagatataccaacatgaaggaatcagttccttttttcgtggcgccttctccaatgttcttcgcggtacagggggtgctttggtgttggtattatatgataaaattaaagaattctttcatattgatattggtggtaggtaatcgggagagtaaattaagaaatacatggatttaacttgttaaacatacaaattacatagctgccatttgcatacattttgatagtgttattgtctgtattttgttaaagtgctagttctgcaataaagcatacattttttcaagaatttaaatactaaaaatcagataaatgtggattttcctcccacttagactcaaacacattttagtgtgatatttcatttattataggtagtatattttaatttgttagtttaaaattctttttatgattaaaaattaatcatataatcctagattaatgctgaaatctaggaaatgaaagtagcgtcttttaaattgctattcatttaatatacctgttttcccatcttttgaagtcatatggtatgacatatttcttaaaagcttatcaatagatgtcatcatatgtgtaggcagaaataagctttgttctatatctcttctaagacagttgttattactgtgtataatatttacagtatcagcctttgattatagatgtgatcatttaaaatttgataatgactttagtgacattataaaactgaaactggaaaataaaatggcttatctgctgatgtttatctttaaaataaataaaatcttgctagtgtgaatacaaaaaaaaaaaaaaaa

SEQ ID NO: 2 - protein, 315 aa, homo sapiens
MHREPAKKKAEKRLFDASSFGKDLLAGGVAAAVSKTAVAPIERVKLLLQVQASSKQISPEARYKGMVDCLVRIPREQGFFSFWRGNLANVIRYFPTQALNFAFKDKYKQLFMSGVNKEKQFWRWFLANLASGGAAGATSLCVVYPLDFARTRLGVDIGKGPEERQFKGLGDCIMKIAKSDGIAGLYQGFGVSVQGIIVYRASYFGAYDTVKGLLPKPKKTPFLVSFFIAQVVTTCSGILSYPFDTVRRRMMMQSGEAKRQYKGTLDCFVKIYQHEGISSFFRGAFSNVLRGTGGALVLVLYDKIKEFFHIDIGGR

SEQ ID NO: 3 - DNA, 1577 bp, Mus musculus
agtgtcgcttgagtgttggtgtggcctgcaggtgtccggttgcccggtcctctgtccaacatgtcgaacgaatcctccaagaagcagtcttcaaagaaggcgctgttcgatccggtgtctttctcgaaggacctgctggccggcggggtcgcggccgcggtgtcgaagacaactgtggcgcccatcgagcgtgtgaagctgctgctgcaggtgcaggcgtcctccaagcagataagccctgaggcgcgctacaagggcatgctggactgcctggtgcgcattcctcgtgagcaaggatttttaagttattggcgtggcaatttggcaaatgttattcgatactttccaacacaagccttaaacttcgcttttaaggacaaatacaaagaacttttcatgtctggtgttaataaagaaaaacagttctggagatggtttctagcaaacctggcttctggaggggctgctggagcaacatccttgtgtgtagtatacccactagattttgccagaacccgattaggtgttgatattggaaaaggtcctgagcagcggcagttcacgggtttgggtgactgcattatgaaaatagccaagtcagatggactgattggtctataccaagggtttggtgtctctgttcagggtatcattgtttaccgagcctcttactttggagcttatgacaccgttaagggcttattgccaaagccaaaggaaaccccatttcttgtctcttttatcattgctcaaatcgtgactacctgttctggaatactctcctatccctttgacacagttagaagacgtatgatgatgcagagtggggaatctgatcggcaatataaaggaaccatagactgctttctgaaaatctaccgtcatgaaggagttcctgccttcttccgtggtgccttctccaacatccttcgtggcacagggggtgctttggtcttggtgttatatgataaaatcaaagagttcctcaacattgatgttggaggtagttcatcaggagattaaattgagaaatgcatatttctaatgtaaaaacatgaaaattacatagctgccatttttatatattttgatagtgtgttactactgtcagtgtctcttacagtatttgttctgcaataaagaaaagatttttttttcaagattttagtattaaaagtcaggacaaaaatttttttcacttagacccagaatcatatattaaggcattttattataggtagtgtatgttacttctgttaaaaaattcttacacttgtgatgaacaccatataatgtgaaatatgaggaagtgtctttaaacttcaatttgcttagtacaacagtaatcccatcttttaggaattgtattgtatgaccaatagttgaaaagttgataatgacttagtgacactatcaaactatttgaaaagtataggttgggctatttgctaatgtttagtcttgctagtgtatataaatctttgaacaagaaatctctggacattagattttgtattctgtatcaataataaagcaagctcaaaactaaaaaaaaaaaaaaaaaaaaaaaaaaaaaa

SEQ ID NO: 4 - protein, 320 aa, Mus musculus
MSNESSKKQSSKKALFDPVSFSKDLLAGGVAAAVSKTTVAPIERVKLLLQVQASSKQISPEARYKGMLDCLVRIPREQGFLSYWRGNLANVIRYFPTQALNFAFKDKYKELFMSGVNKEKQFWRWFLANLASGGAAGATSLCVVYPLDFARTRLGVDIGKGPEQRQFTGLGDCIMKIAKSDGLIGLYQGFGVSVQGIIVYRASYFGAYDTVKGLLPKPKETPFLVSFIIAQIVTTCSGILSYPFDTVRRRMMMQSGESDRQYKGTIDCFLKIYRHEGVPAFFRGAFSNILRGTGGALVLVLYDKIKEFLNIDVGGSSSGD